(19) United States
(12) Patent Application Publication
(10) Pub. No.: US 2015/0216559 A1
(43) Pub. Date: Aug. 6, 2015

(54) PERCUTANEOUS ENDOSCOPIC THERAPY

(71) Applicant: MAYO FOUNDATION FOR MEDICAL EDUCATION AND RESEARCH, Rochester, MN (US)

(72) Inventors: Todd H. Baron, SR., Oronoco, MN (US); Louis-Michel Wong Kee Song, Rochester, MN (US)

(21) Appl. No.: 14/415,392

(22) PCT Filed: Jul. 2, 2013
(86) PCT No.: PCT/US2013/049093

§ 371 Date: Jan. 16, 2015

Related U.S. Application Data
(60) Provisional application No. 61/672,995, filed on Jul. 18, 2012.

(51) Int. Cl. A61B 17/34; A61B 17/02

(52) U.S. Cl. CPC A61B 17/3423; A61B 17/0218; A61B 17/3498; A61B 2017/0225; A61B 2017/3486

(57) ABSTRACT

This document provides methods and materials for performing percutaneous endoscopic therapy (e.g., percutaneous assisted transprosthetic endoscopic therapy). For example, access devices (e.g., inflatable access devices) for performing percutaneous endoscopic therapy are provided.

### CROSS-REFERENCE TO RELATED APPLICATIONS

This application claims the benefit of U.S. Provisional Application Ser. 61/672,995, filed Jul. 18, 2012. The disclosure of the prior applications are considered part of (and are incorporated by reference in) the disclosure of this application.

### BACKGROUND

1. Technical Field This document relates to methods and materials for performing percutaneous endoscopic therapy (e.g., percutaneous assisted transprosthetic endoscopic therapy). For example, this document provides devices (e.g., inflatable devices) for performing percutaneous endoscopic therapy.

2. Background Information

Endoscopy performed through a natural orifice (i.e., per oral or per rectum) may not enable provision of intended therapy at a target site due to inability to access the site. The potential to access the GI tract through a non-natural orifice became apparent when Ponsky and Gauderer described percutaneous endoscopic gastrostomy (PEG) as a technique for gastrostomy feeding tube placement more than 30 years ago. Although the intent was to develop a less invasive alternative to surgical placement, this non-operative procedure opened up a new port of entry through a mature PEG tract for passage of an endoscope into the esophagus, stomach, and duodenum. Subsequently, endoscopic procedures performed through a PEG tract have included retrograde intubation of the esophagus with a small caliber endoscope for rendezvous recanalization of completely obstructed esophageal lumen, placement of self-expandable metal stents in the duodenum, transgastric jejunal feeding tube placement, transgastric ERCP, and placement of a percutaneous jejunostomy tube via a mature PEG site. A descendant of the PEG technique is the direct percutaneous endoscopic jejunostomy (DPEJ) procedure. Similar to PEG, small caliber endoscopes can be passed through a mature PEJ tract to explore the jejunum, albeit to a limited extent.

### SUMMARY

This document provides methods and materials for performing percutaneous endoscopic therapy (e.g., percutaneous assisted transprosthetic endoscopic therapy). For example, this document provides access devices (e.g., inflatable access devices) for performing percutaneous endoscopic therapy.

As described herein, an access device can be configured to be hollow and inflatable and can be dimensioned to be inserted into or across tissue (e.g., skin or muscle) of a patient. Once inserted into a patient, an access device can be inflated in a manner such that an outer diameter of the access device increases. The increasing outer diameter can be configured to provide sufficient force against the patient's tissue so as to overcome tissue resistance and to provide space for the increasing outer diameter. In some cases, an access device can be configured in a manner such that both an outer diameter and an inner diameter of the access device increase upon inflation. The increasing inner diameter can be configured to provide a lumen with additional space to allow a surgeon to introduce one or more endoscopic devices into the patient through the lumen to carry out an endoscopic procedure. Once an endoscopic procedure is completed, the endoscopic devices can be withdrawn from the patient, and the inflated access device can be deflated and withdrawn from the patient.

Having the ability to displace tissue along a surgical path during an endoscopic procedure using an access device provided herein can provide surgeons with additional space to perform the endoscopic procedure and can allow surgeons to remove the access device easily in that additional space can be provided during the withdrawal procedure upon deflation of the inflated access device.

In general, one aspect of this document features an access device for an endoscopic procedure. The access device comprises, or consists essentially of, (a) a tubular member comprising a proximal end region, a distal end region, an inflation chamber, an inner surface defining an inner diameter of the tubular member, and an outer surface defining an outer diameter of the tubular member, wherein the tubular member defines a lumen that extends from the proximal end region to the distal end region and is defined by the inner surface of the tubular member, and (b) an inflation port configured to allow gas to be introduced into the inflation chamber, thereby inflating the access device, wherein the outer diameter of the tubular member increases as the inflation chamber is inflated. The access device can comprise a valve within the lumen. The access device can comprise two or more valves within the lumen. The access device can comprise three valves within the lumen. The valve or the valves can be tricuspid valves. The valve or the valves can be inflatable valves. The inner diameter of the tubular member can be configured to not substantially decrease as the inflation chamber is inflated. The inner diameter of the tubular member can be configured to increase or remain the same as the inflation chamber is inflated. The proximal end region or the distal end region of the tubular member can be flared. The proximal end region and the distal end region of the tubular member can be flared. The access device can comprise a pressure gauge configured to provide information about the pressure within the inflation chamber.

In another aspect, this document features a method of performing an endoscopic procedure within a mammal. The method comprises, or consists essentially of, (a) inserting an access device into the mammal, wherein the access device comprises, or consists essentially of, (i) a tubular member comprising a proximal end region, a distal end region, an inflation chamber, an inner surface defining an inner diameter of the tubular member, and an outer surface defining an outer diameter of the tubular member, wherein the tubular member defines a lumen that extends from the proximal end region to the distal end region and is defined by the inner surface of the tubular member, and (ii) an inflation port configured to allow gas to be introduced into the inflation chamber, wherein the proximal end region is located outside the body of the mammal, and wherein the distal end region is located proximal to a target location within the mammal, (b) introducing gas into the inflation chamber via the inflation port, thereby inflating the inflation chamber, wherein the outer diameter of the tubular member increases as the inflation chamber is inflated, (c) introducing an endoscopic instrument into the mammal via the lumen to carry out the endoscopic procedure, (d) removing the endoscopic instrument from the mammal, (e) releasing gas from the inflation chamber via the inflation port, thereby deflating the inflation chamber, wherein the outer diameter of the tubular member decreases as the inflation chamber is deflated, and (f) removing the access device from the mammal. The mammal can be a human. The access device can comprise a valve within the lumen. The access device can comprise two or more valves within the lumen. The access device can comprise three valves within the lumen. The valve or the valves can be tricuspid valves. The valve or the valves can be inflatable valves. The inner diameter of the tubular member can be configured to not substantially decrease as the inflation chamber is inflated. The inner diameter of the tubular member can be configured to increase or remain the same as the inflation chamber is inflated. The proximal end region or the distal end region of the tubular member can be flared. The proximal end region and the distal end region of the tubular member can be flared. The access device can comprise a pressure gauge configured to provide information about the pressure within the inflation chamber. The gas can be sterile. The gas can be air (e.g., CO₂ or O₂). The target location can be a stomach, colon, small intestine, pancreas, gall bladder, abdominal cavity, or retroperitoneum. The endoscopic instrument can be a flexible endoscopic instrument, a rigid endoscopic instrument, or a laparoscopic instrument.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a side view of an access device according to some embodiments provided herein.

FIG. 2 is a cross sectional view along the A-A line of the access device of FIG. 1.

FIG. 3 is a side view of the access device of FIG. 1 in an inflated state.

FIG. 4 is a side view of an access device having flared end regions according to some embodiments provided herein.

FIG. 5 is a cross sectional view of an access device according to some embodiments provided herein.

FIG. 6 is a cross sectional view of the access device of FIG. 5 during use with an endoscope instrument.

FIG. 7 is an image of a human body showing locations for use of an access device provided herein: a paracolic gutter walled-off pancreatic necrotic collection (A), the gallbladder (B), or the excluded stomach of a Roux-en-Y gastric bypass (C).

### DETAILED DESCRIPTION

This document provides methods and materials for performing percutaneous endoscopic therapy (e.g., percutaneous assisted transprosthetic endoscopic therapy). For example, this document provides access devices (e.g., inflatable access devices) for performing percutaneous endoscopic therapy.

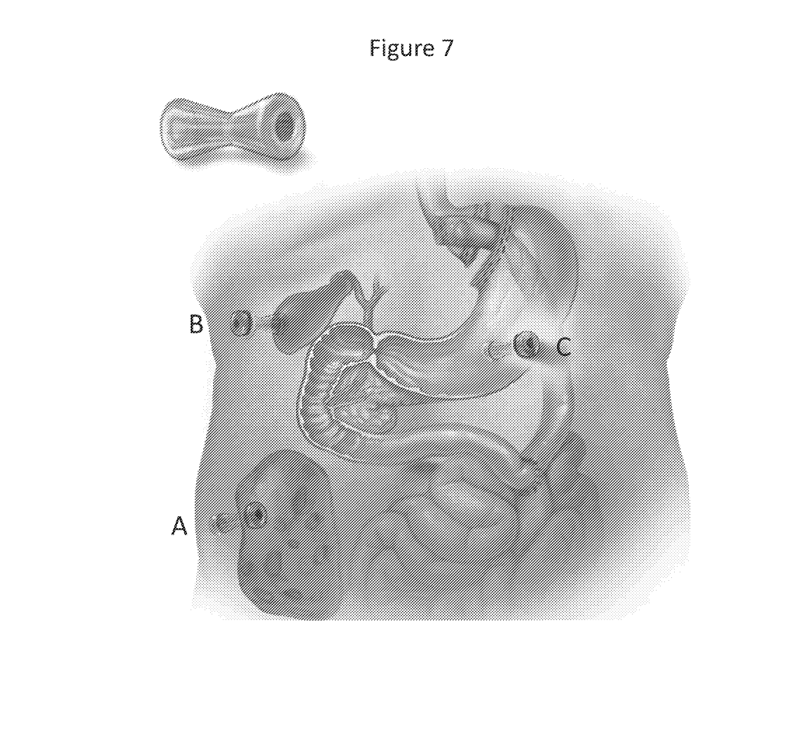

An access device provided herein can be configured to have a tubular member defining an inner lumen and having a proximal end region, a central region, and a distal end region. The proximal end region can be configured to remain outside a patient's body during use. The distal end region can be configured to be placed within a patient's body during use. In some cases, the distal end region of an access device provided herein can be configured to extend to a desired location within the patient. For example, an access device provided herein can be configured such that the proximal end region of the access device remains outside a patient's body while the distal end region is positioned within the patient's stomach. Examples of other desired locations within a human body include, without limitation, the pancreas, gallbladder, small intestine, colon, intra-abdominal abscesses or other collections, and retroperitonal collections. In some cases, an access device provided herein can be positioned as shown in FIG. 7, can be positioned into the colon (e.g., right colon) to allow passage of an endoscope for treatment of an ileal stricture, or can be positioned into the jejunum to allow passage of an endoscope for treatment of mid-jejunal stricture.

Once in position within a mammal (e.g., a human), an access device provided herein can be inflated such that an outer diameter of the access device increases. In some cases, inflating an access device provided herein can result in an increased outer diameter with little or no change to the length of the inner diameter. The increasing outer diameter can be configured to provide sufficient force against the patient's tissue so as to overcome tissue resistance and to provide space for the increasing outer diameter. In some cases, an access device can be configured in a manner such that both an outer diameter and an inner diameter of the access device increase upon inflation. The increasing inner diameter can be configured to provide additional space within the lumen to allow a surgeon to introduce one or more endoscopic devices into the patient through the lumen to carry out an endoscopic procedure. Once an endoscopic procedure is completed, the endoscopic devices can be withdrawn from the patient, and the inflated access device can be deflated and withdrawn from the patient. In some cases, an access device provided herein can include one or more valves within the lumen (e.g., one, two, three, four, five, or more valves). Such valves can be configured to reduce or prevent the flow or escape of material (e.g., bowel or stomach material) from the patient through the lumen of the access device. In some cases, the one or more valves can allow an endoscopic device to be advanced within the lumen from the proximal end region to the distal end region while reducing or preventing the flow or escape of material from the patient through the lumen of the access device. Any appropriate valve can be used to make an access device provided herein. For example, tricuspid valves, anti-reflux valves (see e.g., U.S. Pat. No. 6,682,503), triple-petal structures (see, e.g., U.S. Patent Application Publication No. 2009/013807), Tuohy Borst valves (see, e.g., U.S. Pat. No. 5,195,980), flutter valves, valve sleeves, or self-sealing membranes can be placed within a lumen of an access device provided herein. In some cases, an access device provided herein can include one or more valves formed with two or more (e.g., three, four, or more) leaflets. When two or more leaflets are used to form a valve, the leaflets can be in the same plane along the length of the tubular member or can be offset to be in different planes provided that the leaflets form a valve. When in different planes, the leaflets can be larger than those used when the leaflets are in the same plane. In some cases, the valves of an access device provided herein can be inflatable. For example, the inflation chamber of an access device provided herein can extend into the inner portions of valves such that the valves inflate upon inflation of the access device. In some cases, one or more of the valves can be inflated separately from inflation of the access device.

In some cases, an access device provided herein can include a cap or plug (e.g., a balloon plug) for the lumen in place of or in addition to one or more valves within the lumen to reduce or prevent the flow or escape of material (e.g., bowel or stomach material) from the patient through the lumen of the access device.

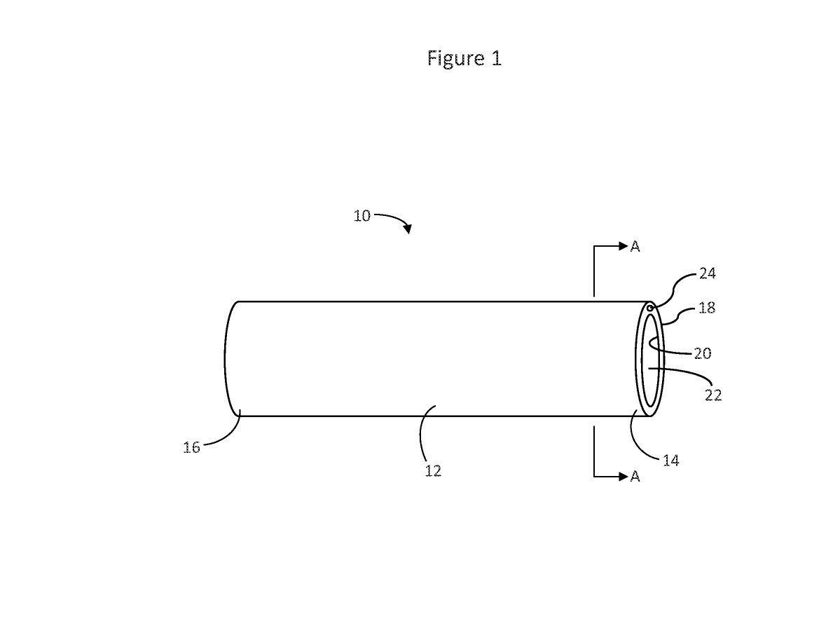

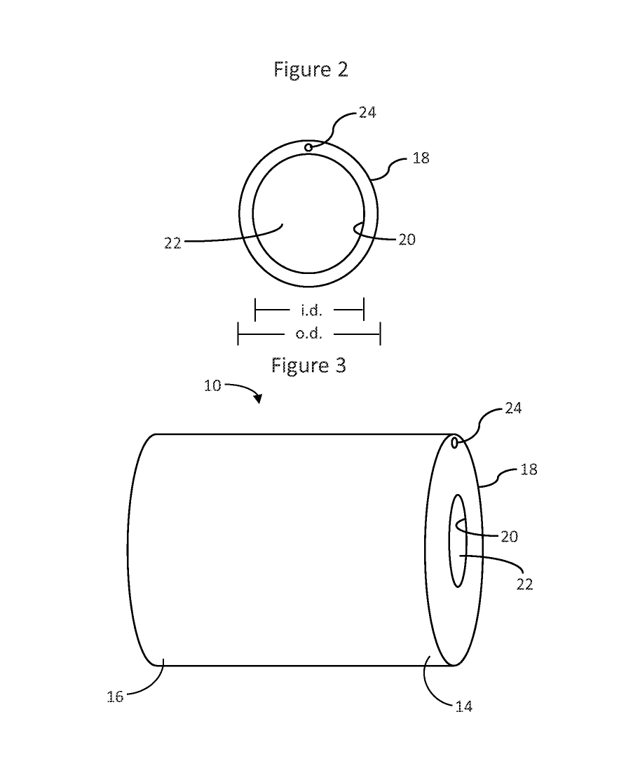

With reference to FIGS. 1 and 2, an access device 10 can include a tubular member 12 having a proximal end region 14 and a distal end region 16. Tubular member 12 can define a lumen 22. In some cases, lumen 22 can extend the entire longitudinal length of assess device 10.

In addition, access device 10 can have an outer surface 18 and an inner surface 20. As shown in FIG. 2, outer surface 18 can define an outer diameter (o.d.) of access device 10, and inner surface 20 can define an inner diameter (i.d.) of access device 10.

In some cases, proximal end region 14 of assess device 10 can include an inflation port 24. Inflation port 24 can be configured to receive an inflation device or tubing designed to deliver gas to an inner chamber of access device 10. In some cases, inflation port 24 can be a self-sealing port such that access device 10 remains inflated once inflated and the inflation source is removed. In some cases, a cap or plug separate from or integral with access device 10 can be used to open and close inflation port 24. In some cases, inflation port 24 can be configured as a valve. For example, inflation port 24 can be a check valve, ball valve, or flutter valve. In some cases, access device 10 can include a gauge configured to provide information about the pressure level within a chamber of access device 10.

As gas is delivered to an inner chamber of access device 10, access device 10 inflates. In some cases, inflating access device 10 can result in an increasing outer diameter as shown in

FIG. 3 with little or no increase in the inner diameter. In such cases, the material of outer surface 18 can be flexible, and the material of inner surface 20 can be rigid. For example, the material of outer surface 18 can be flexible, and the material of inner surface 20 can be rigid.

In some cases, inflating access device 10 can result in both an increasing outer diameter and an increasing inner diameter. In such cases, the material of outer surface 18 and the material of inner surface 20 can be flexible. In some cases, the material of outer surface 18 can be slightly more flexible than the material of inner surface 20. In some cases, one or more support structures can be located within the inflation chamber of access device 10 to connect outer surface 18 to inner surface 20 such that inflation of access device 10 causes both an increasing outer diameter and an increasing inner diameter as opposed to an increasing outer diameter and a decreasing inner diameter. Such support structures can be rigid and can be spaced (e.g., evenly spaced) within the inflation chamber of access device 10 such that both the outer diameter and the inner diameter increase as access device 10 is inflated.

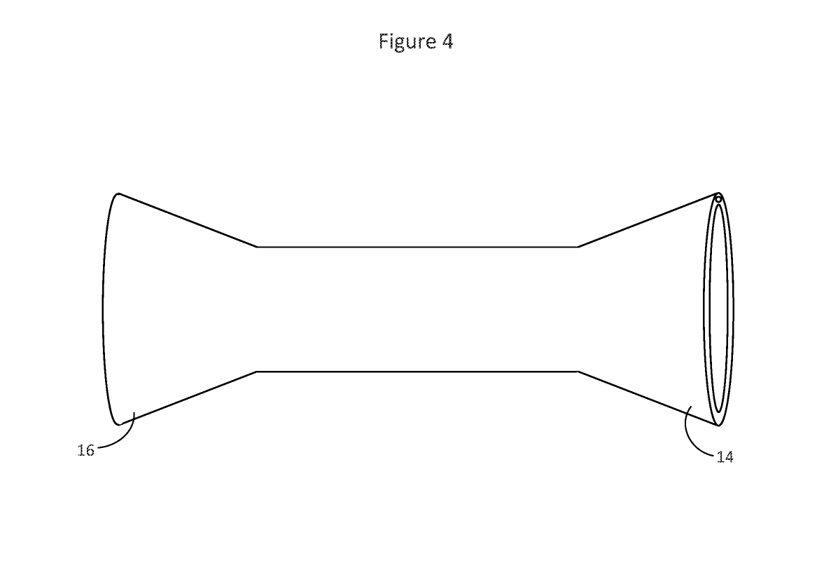

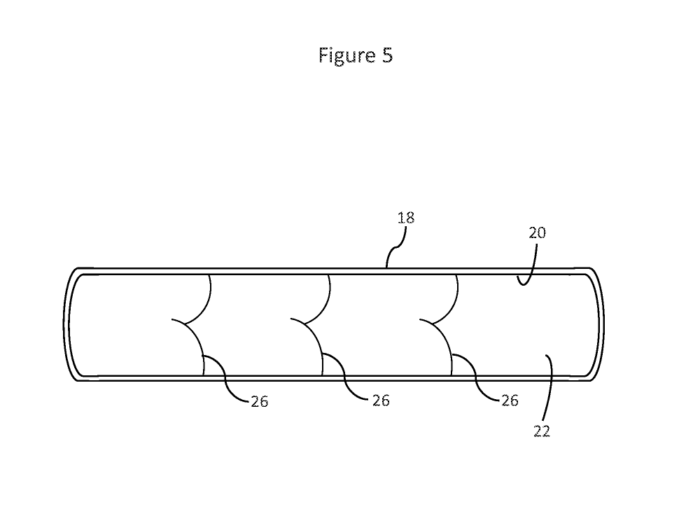

In some cases, one or both of proximal end region and distal end region can be flared. For example, with reference to FIG. 4, an access device provided herein can have a flared proximal end region 14 and a flared distal end region 16.

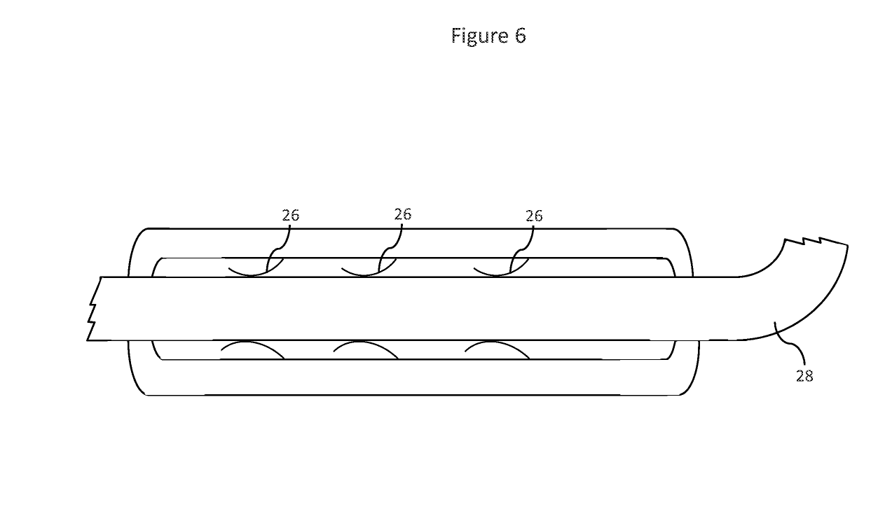

With reference to FIGS. 5 and 6, an access device provided herein can include one or more valves 26 within lumen 22. Such valves can be configured to reduce or prevent the flow or escape of material (e.g., bowel or stomach material) from the patient through the lumen of the access device. In some cases, one or more valves 26 can allow an endoscopic device 28 to be advanced within lumen 22 from the proximal end region to the distal end region while reducing or preventing the flow or escape of material from the patient through lumen 22 of the access device. Any appropriate valve can be used to make an access device provided herein. For example, tricuspid valves, anti-reflux valves (see e.g., U.S. Pat. No. 6,682,503), triple-petal structures (see, e.g., U.S. Patent Application Publication No. 2009/013807), Tuohy Borst valves (see, e.g., U.S. Pat. No. 5,195,980), flutter valves, valve sleeves, or self-sealing membranes can be placed within a lumen of an access device provided herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An access device for an endoscopic procedure, wherein said access device comprises:
(a) a tubular member comprising a proximal end region, a distal end region, an inflation chamber, an inner surface defining an inner diameter of said tubular member, and an outer surface defining an outer diameter of said tubular member, wherein said tubular member defines a lumen that extends from said proximal end region to said distal end region and is defined by said inner surface of said tubular member, and (b) an inflation port configured to allow gas to be introduced into said inflation chamber, thereby inflating said access device, wherein said outer diameter of said tubular member increases as said inflation chamber is inflated.

2. The access device of claim 1, wherein said access device comprises a valve within said lumen.

3. The access device of claim 1, wherein said access device comprises two or more valves within said lumen.

4. (canceled)

5. The access device of claim 2, wherein said valve is tricuspid valves.

6. The access device of claim 2 wherein said valve is inflatable valves.

7. The access device of claim 1, wherein said inner diameter of said tubular member does not substantially decrease as said inflation chamber is inflated.

8. The access device of claim 1, wherein said inner diameter of said tubular member increases or remains the same as said inflation chamber is inflated.

9. The access device of claim 1, wherein said proximal end region or said distal end region of said tubular member is flared.

10. (canceled)

11. The access device of claim 1, wherein said access device comprises a pressure gauge configured to provide information about the pressure within said inflation chamber.

12. A method of performing an endoscopic procedure within a mammal, wherein said method comprises:
(a) inserting an access device into said mammal, wherein said access device comprises:
(i) a tubular member comprising a proximal end region, a distal end region, an inflation chamber, an inner surface defining an inner diameter of said tubular member, and an outer surface defining an outer diameter of said tubular member, wherein said tubular member defines a lumen that extends from said proximal end region to said distal end region and is defined by said inner surface of said tubular member, and
(ii) an inflation port configured to allow gas to be introduced into said inflation chamber,
wherein said proximal end region is located outside the body of said mammal, and wherein said distal end region is located proximal to a target location within said mammal,
(b) introducing gas into said inflation chamber via said inflation port, thereby inflating said inflation chamber, wherein said outer diameter of said tubular member increases as said inflation chamber is inflated,
(c) introducing an endoscopic instrument into said mammal via said lumen to carry out said endoscopic procedure,
(d) removing said endoscopic instrument from said mammal,
(e) releasing gas from said inflation chamber via said inflation port, thereby deflating said inflation chamber, wherein said outer diameter of said tubular member decreases as said inflation chamber is deflated, and
(f) removing said access device from said mammal.

13. The method of claim 12, wherein said mammal is a human.

14. The method of claim 12, wherein said access device comprises a valve within said lumen.

15-17. (canceled)

18. The method of claim 14, wherein said valve is or said valves are inflatable valves.

19. The method of claim 12, wherein said inner diameter of said tubular member does not substantially decrease as said inflation chamber is inflated.

20. The method of claim 12, wherein said inner diameter of said tubular member increases or remains the same as said inflation chamber is inflated.

21. The method of claim 12, wherein said proximal end region or said distal end region of said tubular member is flared.

22. The method of claim 12, wherein said proximal end region and said distal end region of said tubular member are flared.

23. The method of claim 12, wherein said access device comprises a pressure gauge configured to provide information about the pressure within said inflation chamber.

24-25. (canceled)

26. The method of claim 12, wherein said gas is CO₂ or O₂.

27. (canceled)

28. The method of claim 12, wherein said endoscopic instrument is a flexible endoscopic instrument, a rigid endoscopic instrument, or a laparoscopic instrument.
